(19) United States
(12) Patent Application Publication
(10) Pub. No.: US 2006/0188573 A1
(43) Pub. Date: Aug. 24, 2006

(54) Composite materials and particles

(76) Inventor: Anna Imberg, Uppsala (SE)

Correspondence Address: YOUNG & THOMPSON, 745 SOUTH 23RD STREET, 2ND FLOOR, ARLINGTON, VA, 22202, US

(21) Appl. No.: 11/336,819

(22) Filed: Jan. 23, 2006

Related U.S. Application Data
(63) Continuation-in-part of application No. 10/560,165.

(30) Foreign Application Priority Data
Jun. 10, 2003 (SE) 0301722-5

(51) Int. Cl. A61K 9/22

(52) U.S. Cl. 424/468

(57) ABSTRACT

A method or concept for producing a composite material having at least an amphifilic (i.e., surface-active) and polymer component, and the composite material formed thereby, includes providing a mixture of at least one each of polymer and amphifilic compound in a volatile solvent or solvent mixture and providing a phase diagram of the chemical system describing how the components of the chemical system interact in thermodynamic stable phases as a function of temperature, concentration and pressure. The polymer should be a homopolymer, a random block copolymer or a mixture thereof, preferably biodegradable. The amphifilic compound can form a bilayer-containing phase. The solvent is removed from the mixture by a process selected from the phase diagram considering the desired final composite material, whereby a material is formed. The composite material is useful for applications such as encapsulation of therapeutically active components or for applications such as surface coating.

### FIELD OF THE INVENTION

The present invention relates to a method, based on a phase diagram approach, where a multi-component chemical system is employed for formation of biocompatible lipid/polymer composite materials, in particular hybrid particles (micro-/nano-particles) or implants. The particles are suitable for systemic and local delivery, of therapeutic active components (peptide, protein, vaccines, nucleic acids or polynucleotides, DNA, RNA), directly within or upon body tissues. It also relates to composite materials (in particular hybrid particles) themselves.

### BACKGROUND OF THE INVENTION AND DESCRIPTION OF THE RELATED ART

A general problem in the medical field is the design of pharmaceuticals such that the active substance can be administered to the patient in the most efficient way possible. One common design method is to provide encapsulated substances such that the release thereof can be controlled over time, so called sustained or delayed release. Encapsulation can also be used to protect sensitive substances from degradation in the hostile environment in the gastric region. By using composite materials to deliver active substances it is often possible to achieve desirable properties of the formulation, e.g. high efficiency of encapsulation, or good protection of active substances from contact with a hostile environment. Biodegradable polymers are together with lipids, that have the ability to self-assembly and to form membranes, two examples of components used for the creation of drug-delivery systems applied in more or less water-rich environments.

The biodegradable polymer, poly(D,L-lactide-co-glycolide) (PLG) has, during the last decade, been widely adopted in the medical field (i.e. for sutures etc.), and certain variants are approved by the FDA. PLG has been used in injectable gels, in situ forming implants, and microparticles. A common characteristic of these different formulations is that acid environments are created as a consequence of the polymer degradation into lactic acid and glycolic acid. In many cases, proteins are sensitive to pH reduction. Therefore, the usefulness of such systems in pharmaceutical applications is limited.

Lipids, with the ability to self-assemble into monolayer- or bilayer-containing structures, with length-scales in the colloidal domain, are interesting for drug delivery applications of hydrophilic, hydrophobic and amphiphilic components. Examples of interesting structures, for drug delivery applications, are cubic-, sponge-, hexagonal- and lamellar phases. Which one of these structures that will form depends on some specific properties of the amphiphilic lipid (i.e. geometrical shape and spontaneous curvature) and the volume fractions of water and lipid. One example of a polar lipid is Monoolein (glyceryl-mono-oleate), a metabolite during fat digestion in the upper intestine, which forms a cubic bicontinuous phase in excess water. The cubic phase, spontaneously formed in aqueous environments, have a thermodynamically stable structure, wherein the lipid bilayer separates two networks of bilayers. The bicontinuous cubic phase(-s) consist(s) of one lipid bilayer that extends in all three dimensions. Two separate “water channel systems” are present within the phase. The size of the water-channels varies with composition. For instance, the diameter of the water channels in the most swollen cubic phase (i.e. a cubic phase consisting of 60/40 weight % MO/water) is approximately 50 Å.

For drug delivery, the cubic phase is especially interesting as a drug delivery candidate with a structure suitable for (controlled) delivery of sensitive biologically active components. Moreover, the cubic phase can be used for site specific targets. The incorporated hydrophilic substances are, in vitro, expected to be released according to a square root of time-release kinetics.

Unsuccessful attempts to find suitable systems for creation of lipid/polymer hybrid particles have been reported in the literature (Johansson et al in J. Phys. Chem. B 2001, 105, 12157-12164). In this study, the solvent, 1-methyl-2-pyrrolidinone (NMP), preferred water to the polymer-rich phase, a fact that made homogenization in pure water impossible. Therefore it was not possible to produce, from emulsion techniques, lipid polymer composite particles of the kind here described.

The following patents and patent applications are cited since they include the simultaneous use of the same lipid and polymer as in the present invention.

U.S. Pat. No. 6,277,413 issued in the name of Sankaram et al, describes how the release rates, of encapsulated substances can be controlled by varying the ratio between lipid/polymer in lipid/polymer particles.

U.S. Pat. No. 6,488,952 issued in the name of Kennedy et al, describes how a multiparticulate system, for injection, deposition or implantation, can be formed by incorporation of polymer micro-particles in a hydrogel (i.e. cubic phase).

International Patent Application No. WO02/19988 A2 issued in the name of Bodmeier et al, describes how sustained release particles can be formed, from liquid components. The particles contain domains with another density in order to achieve the desired sustained release properties.

International Patent Application No. WO99/47588 issued in the name of Dawson et al, describes a method for preparation of polymer microparticles, over a wide range of preparation conditions, by using an emulsifier (i.e. MO).

### SUMMARY OF THE INVENTION

The object of the present invention is to facilitate the formation of certain kinds of composite materials, in particular composite lipid/polymer hybrid particles, with well-separated lipid domains dispersed in the biodegradable polymer matrix, for drug delivery of sensitive biologically active substances, as well as a route for formation of such materials and lipid/polymer hybrid particles. The lipid domains should preferably swell and form a cubic phase (or another liquid crystalline phase or monolayer-containing phase given from the binary lipid (or another amphiphilic molecule)/water phase diagram) in excess water.

This object is achieved by selecting a chemical system with a suitable phase behavior from a phase diagram that describes the phase behavior of the chemical system and following a novel route for formation of lipid/polymer composite materials, such as composite particles, implants, surface coatings etc, by shifting the thermodynamic equilibrium point of the system in a desired direction based on the phase diagram.

In the present invention, a volatile solvent that is not completely miscible with water, i.e. the solubility of the solvent in water is approximately 8% by weight, has been chosen. As this solvent is not completely miscible with water, it is suitable for particle formation by emulsification methods. The solvent's partial miscibility in water has been used to set the preferred kinetics (i.e. to make it possible to study the phase separation) for the lipid domain formation.

The invention involves the provision of how to select a combination of components, that together form a chemical system that exhibits a phase behavior that enables formation of such particles and materials. The method of making composite materials and in particular particles, according to the invention, is defined in claim 1 and is based, for instance, on an emulsification method, where a proper amount of the polymer/lipid/solvent phase(-s) is homogenized into a second solvent in which the polymer is at least only moderately soluble and preferably insoluble. In another aspect, other methods can also be used, e.g. spray-drying, atomization and super critical fluid techniques.

A selected composition range is employed, given by the phase behavior, from which, materials and/or particles can be formed by controlled solvent removal.

In particular, the present invention makes use of the functionality (e.g. the water-swelling properties and simultaneous formation of monolayer- or bilayer structure(s)) of the lipid, for drug delivery.

Preferably, water channels, which are present in the cubic phase, are used for encapsulation of hydrophilic substances.

Alternatively, the membrane formed, i.e. the monolayer- or bilayer structure, is utilized for encapsulation of hydrophobic substances.

Furthermore, according to the present invention the monolayer or the bilayer forming component build the cubic phase, (or another desired phase), that is used for protection and encapsulation of membrane bound proteins, and other suitable amphiphilic therapeutic active substances.

The lipid is suitably used for increasing the mucoadhesion of the formulation.

Preferably, the formulation according to the invention is used for controlled/sustained release applications.

Suitably, the kinetics for phase separation between the lipid and the polymer is adjusted by using different molecular weights and types of the components (i.e. lipids, polymers, solvents).

In accordance with the present invention it is possible to use any polar lipid (or other amphiphilic molecule) with the ability to form the desired liquid crystalline or monolayer-containing phase in aqueous environments. Examples of such polar lipids are monoelaidin, phosphatidylethanolamine, phosphatidylcholine, phospholipids, PEGylated phospholipids, sfingolipids, cholesterol, and brain- or skin lipids.

The lipid/polymer hybrid particles should preferably contain at least one biodegradable polymer. The polymer can be a homopolymer, a random block copolymer or a mixture thereof. The polymer should be partially or completely soluble in organic solvents and not be completely soluble in water.

Examples of biodegradable polymers usable for formation of lipid/polymer composite materials and hybrid particles, are e.g. poly(lactide), poly(glycolide), poly(p-dioxanone), poly(caprolactone), polyhydroxyalkanoate, polypropylenefumarate, polyorthoesters, polyphosphateesters and polyanhydrides, for formation of lipid/polymer hybrid particles.

The lipid/polymer hybrid particles are suitably formed with lipid domains dispersed into the polymer-matrix, or with a lipid (or polymer) core within a polymer (or lipid) shell.

As indicated above, not only particles can be formed, but also e.g. solid implants and semi-solid, gel-like matrices. By using a water-insoluble polymer the former can be formed and using a slightly water-soluble polymer the latter can be formed.

In another aspect of the present invention, also very porous particles can be created from the same chemical system by means of the following two-step concept. In the first step, lipid/polymer composite particles are created according to the detailed description given in Example 1. In the second step, the swollen lipid domains are washed out by a solvent that acts as a solvent for the lipid but not for the polymer. In this way, very porous polymer particles can be created. Such particles might, after addition of a therapeutic active component, be useful for inhalation applications. The final degree of porosity depends on the amount of lipid present. It should be possible to create particles with a degree of porosity of approximately 5-70%.

### BRIEF DESCRIPTION OF THE DRAWINGS

The present invention will be better clarified upon reading the detailed description with reference to the drawings, in which

FIG. 1 is a plot of the phase diagram of the quaternary lipid/polymer/solvent/water system at constant temperature (20° C.] and pressure [1 atm];

FIG. 2 is a schematic presentation of the cubic phase(-s);

FIG. 3 is a schematic description of the present emulsification method;

FIG. 4 is a confocal laser scanning probe microscopy CLSM picture of the lipid/polymer hybrid particles;

FIG. 5 is a confocal laser scanning probe microscopy CLSM picture of the polymer reference particles;

### DETAILED DESCRIPTION OF THE INVENTION

Briefly, the invention is directed to design of appropriate manufacturing methods for formation of, for example, formulations (or other preparations) based on a phase diagram approach. Phase diagrams define how the components of the chemical system interact, in thermodynamic stable phases, as a function of concentration, temperature, and pressure. The main idea is that phase diagrams, that describe how different components interact in stable structures (phases) that are spontaneously formed as phase diagram express the behavior at thermodynamic equilibrium, are used for design of appropriate manufacturing methods. As pharmaceutics often contain different components (active substances and other substances required for creation of an appropriate formulation) and stability requirements are needed, the phase-diagram approach here described for preparation and evaluation of, for instance pharmaceutical preparations, is efficient.

Below the phase behavior is further presented as well as the particle formation process. The most important properties of the chemical system are highlighted and examples for different applications are given.

The description and examples below serve to illustrate the invention in detail, but the chemical system illustrated in the examples is only one way of carrying out the invention. Numerous variations exits within the scope defined by the claims, and the skilled man will be able to obtain variations of the invention without exercising inventive skill.

Although reference is made mainly to particle formation in the examples, it is to be understood that the invention has much broader applicability, and that it is possible to make surface coatings, solid implants and semi-solid, gel-like matrices implants, etc, by using the basic concept given by the invention.

As used herein the term “solvent” can be not only a single chemical entity, such as ethanol, cyclohexane or water, but also mixtures of one or more solvents.

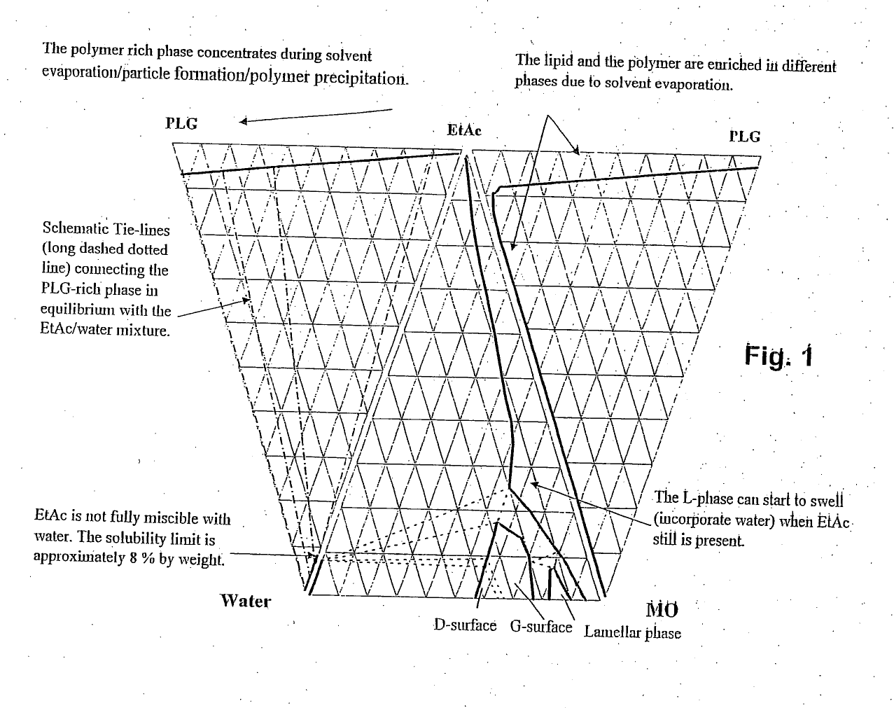

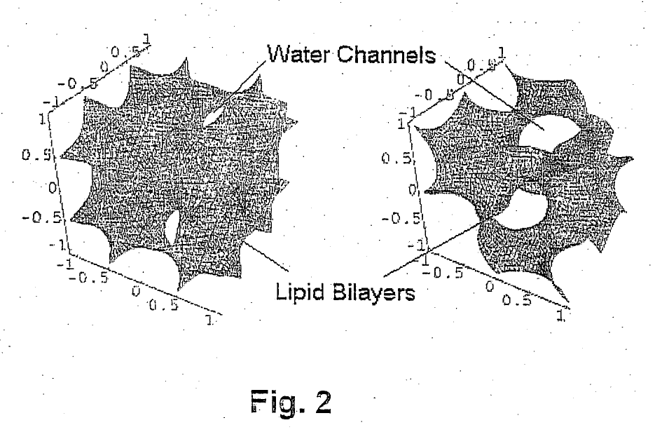

FIG. 1 schematically illustrates a ternary phase diagram, showing the various phases of interest for the invention. In particular, the location of cubic phase is presented therein, and the structure of the cubic phase, where both the bilayer and the water channels are further presented, is given in FIG. 2. It should be noted that the system, presented in FIG. 1, only is an example of the present invention, i.e. other components can be used as well.

This description of the invention is based on the phase behavior of the quaternary Poly(D,L-lactide-co-glycolide) (PLG)/monoolein (MO)/ethyl acetate (EtAc)/water system, which has been determined and characterized by the inventor. In FIG. 1, the phase diagram of the quaternary system is given.

The phase diagram of a quaternary system can be viewed as a tetrahedron. Briefly, FIG. 1 shows the phase diagrams of the ternary subsystems EtAc/MO/water, EtAc/MO/PLG and MO/PLG/water. Even though a systematic survey of the inside of the tetrahedron has not been undertaken, a pretty good understanding of the phase behavior of the quaternary system can be obtained by taking into consideration the phase behaviors of the subsystems (i.e. the surfaces of the tetrahedron), which all are further explained below. Note, that the type of PLG used for determination of the phase diagram, given in FIG. 1, has polyethylene glycol segments incorporated on the poly(lactide-co-glycolide) backbone. Qualitatively, the phase behavior is the same when using the pure poly(lactide-co-glycolide) polymer. However, the critical EtAc concentrations, i.e. the amount of EtAc needed to form a true one-phase solution of MO and PLG in the EtAc/MO/PLG system, varies with polymer specific properties, such as type and molecular weight. As briefly mentioned, two different polymers have been used. One is a classical PLG and the other is a more recently developed PEG-PLG. The idea behind using PEG-anchored PLG is that the PEG-segments are assumed to be present at the interface between MO and PLG and therefore the surface energy of the interface is lowered. PEG is just one example of a possible polymer-modification. Examples of other groups/components that can be used for modification of the polymer are polyvinylalcohol, polar uncharged (or charged) lipids or amphiphilic compounds.

The phase behavior reveals that MO and PLG do not mix at all. In fact, they separate in two different phases since their interaction is repulsive. The phase separation can be reduced by addition of a suitable solvent, e.g. EtAc, with the ability to dilute the repulsive interaction between PLG and MO. At high EtAc contents, MO and PLG coexist, without phase separation, in one common phase denoted the L-phase in FIG. 1. Since EtAc is somewhat soluble in water, water addition consequently induces phase separation of the L-phase as a result of solvent removal.

For applications such as formation of particles with lipid domains, the one-phase solution found at high solvent content, is preferably used as the starting mixture. Briefly, particles with lipid domains are formed by emulsifying the L-phase into an aqueous solution and as a consequence of the above mentioned phase separation the desired particles are formed. A further description of the particle formation process is given below.

For application purposes, it is of main importance to control the kinetics of the phase separation, between MO and PLG. Thus different properties affecting the strength of the phase separation can be varied. The phase separation, i.e. segregation, is affected by the properties of the polymer (e.g. molecular weight, amount of PEG and PEG-length). In general, high molecular weight and low PEG-content of the polymer are likely to yield a strong phase separation. PEG is considered to lower the surface energy between the lipid and the polymer and hence affecting the kinetics (i.e. the strength) of the phase separation. In order to adjust the phase separation between the lipid and the polymer, it is possible to use different lengths of the PEG chains and different degrees of PEGylation. In general, increasing the degree of PEGylation reduces the strength of the phase separation between MO and PLG.

The pure Poly(lactide-co-glycolide), purchased from Boehringer Ingelheim Pharma GmbH & CO. Fine Chemicals (Ingelheim am Rhein, Germany), has a molecular weight of 50 000 calculated as a weight mean value. The corresponding number mean value is 11 000.

The PEG-PLG, obtained from Birmingham Polymers Inc. contains 70% PLG and 30% PEG 1000 (lot number D99164). The inherent viscosity was measured to be 0.45 dl/g.

Phase Behavior of the EtAc/MO/PLG System

The phase separation between MO and PLG can be reduced by addition of a proper solvent, like for instance EtAc. Therefore, at high EtAc-contents, only one phase, denoted the L-phase, exist. The solvent prefers MO to PLG. Hence, the system can be regarded as somewhat asymmetric. The EtAc critical concentration, i.e. the lowest concentration EtAc present in order to obtain one phase for all compositions of MO and PLG, is approximately 99% for the pure PLG polymer and 88% for the PEGylated polymer.

Phase Behavior of the EtAc/PLG/Water System

Almost all water forms an EtAc/water mixture that coexists with a PLG-rich phase, see FIG. 1. The slope of the tie-lines indicate how the polymer rich phase will concentrate, as a consequence of solvent removal, during particle formation.

Phase Behavior of the EtAc/MO/Water System

The phase diagram of the ternary EtAc/MO/water system, shown in FIG. 1, contains several one-phase regions; one liquid phase (L-phase), two different bicontinuous cubic phases (i.e. the cubic phases are the G- and D-surface) and one lamellar phase (L_{α}). In between the different one-phase regions, two- and three-phase regions are present.

Note how the water content of the L-phase increases at approximately 35% EtAc by weight. Most likely, this is due to MO aggregation.

Particle Formation

Lipid/polymer hybrid particles were formed according to the following procedure, further described in Example 1, where a liquid phase (L-phase) was emulsified into a water-rich phase.

The following procedure was used for formation of the L-phase: The lipid probe, Lissamine™ rhodamine B 1,2-dihexadecanoyl-sn-glycero-3-phosphoethanolamine triethylammonium salt (rhodamine DHPE), was first dissolved in 95% ethanol. The Rhodamine-DHPE solution was then mixed with MO. The mixture was left at 50° C. until the ethanol had evaporated. Polymer and solvent were then added, so the final sample contained just one true solution, denoted the L-phase in FIG. 1.

The L-phase, used for formation of reference particles, contained the same amounts of DHPE-probe, PLG and EtAc as the L-phase used for formation of lipid/polymer/hybrid particles. The only difference was that the latter also contain some MO.

Particles were formed by emulsifying 0.1 ml L-phase (the composition of the L-phase is given in Example 1) into 1.16 gram of the outer water-rich phase, which consisted of 0%, 4%, 6%, or 8% EtAc in water. Both phases were room-temperatured when the emulsions were created by means of a Polytron mixer (Kinematica AG, PT300) working for 3 minutes at 4000 rpm.

Particle Formation from Phase Behavior

According to the phase diagram, given in FIG. 1, the schematic tie-lines of the system reveal that both the PLG/EtAc and the MO/EtAc mixtures can be in equilibrium with an EtAc/water mixture. The slope of the tie-lines indicates that it is easy to create a stable emulsion, consisting of droplets of the L-phase dispersed in the outer phase, from this chemical system. Hence, the system is suitable to use for formation of lipid/polymer hybrid particles by means of an emulsion technique.

Solid particles are formed as the polymer in the emulsion droplets of the L-phase “precipitates” during removal of the polymer solvent.

The removal of solvent, from the emulsion droplets, occurs since the solvent is somewhat miscible in water. Hence, the solvent will immediately diffuse out to the outer phase as a small amount of the L-phase is emulsified into an outer phase consisting of pure water. As times goes by, the volatile solvent will evaporate from the system. Consequently, the polymer precipitates and form spherical polymer particles as the solvent leave the droplets.

At the same time, the lipid separates from the polymer phase and forms lipid domains within the polymer particles. As water enters inside the particles, the lipid domains begin to swell. First a liquid phase is formed according to the phase behavior given in FIG. 1. Due to further swelling, i.e. further incorporation of water; the lipid forms a cubic G-structure. The cubic G-structure is transformed to a cubic D-structure as a result of additional swelling. The latter exists in equilibrium with excess water, according to the phase diagram. Later on, the swollen lipid domains are rejected from the polymer matrix.

Important Properties of the Chemical System and its Components

One of the most important properties of the system, from a particle formation point of view, is that the L-phase can be in thermodynamic equilibrium with an EtAc/water mixture. The two phases are, in fact, coexisting when particles are prepared by means of emulsion methods. Therefore, this system is “stable and easy to use” for applications such as particle formation by emulsion techniques. Schematic tie-lines, connecting the two phases at equilibrium, are shown in the EtAc/PLG/water phase diagram. The slopes of the tie-lines illustrate how the PLG/EtAc phase changes its composition during particle formation (i.e. during the evaporation of solvent from the system).

Another suitable property of the chemical system is that the volatile solvent, to some extent, is miscible in water. Thus making it possible to study the kinetics for lipid/polymer phase separation (i.e. the rate at which EtAc leaves the particles) by varying the composition of the outer phase.

Another important property of the chemical system that also affects the kinetics of phase separation between lipid and polymer is that the lipid begins to swell (i.e. incorporate water) while some EtAc still is present (i.e. hence affecting the diffusion of MO).

In summary, from a particle formation point of view, the phase behavior of the quaternary system according to the invention favour the particle formation; one important property of the system is that the lipid begins to swell (incorporate water) while ethyl acetate is still present and finally forms a bicontinuous cubic phase (that interestingly also is formed at a relatively high EtAc contents, see FIG. 1). Another important property is that EtAc is not completely miscible with water. A fact that makes it possible to emulsify a polymer-rich phase (containing PLG/MO/EtAc) into a water-rich phase since the polymer-rich phase (that also contains some MO and EtAc) can be in thermodynamic equilibrium with a very water-rich phase. Also, due to the fact that EtAc, to some extent, is soluble in water (the solubility limit is close to 8% EtAc in water). By varying the composition of the outer phase the rate at which EtAc leaves the particles can be varied, by varying the composition of the outer phase.

The lipid and the polymer exhibit a strong phase separation. By choosing a proper solvent, the interaction is reduced so that only one liquid phase (L-phase) appears. By emulsifying the L-phase into aqueous solutions lipid-polymer composite particles are formed.

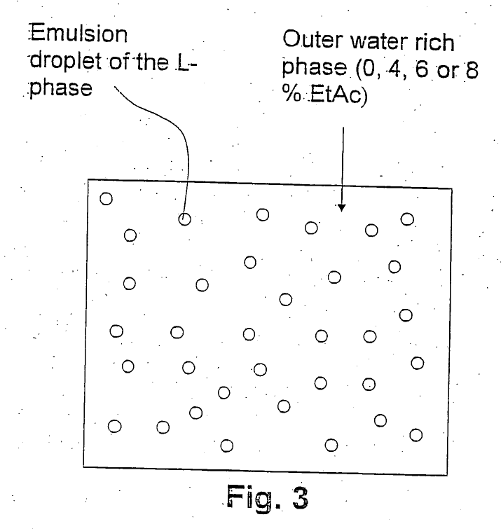

Characterization, of the lipid/polymer composite particles thus obtained, by means of confocal laser scanning microscopy, revealed that distinctive water-swelling lipid domains appeared inside the particles when the polymer concentration was increased as the solvent diffused out of the particles. Finally, the swollen lipid domains left the polymer matrix. FIG. 3 illustrate the formation of particles and the process is further described below, see Example 1.

The quaternary system, on which the invention relies, exhibits a phase behavior that gives a route for formation of lipid/polymer hybrid particles. The particles can be formulated to yield different properties (release rates, domain-sizes, structures) depending on how the particles are created. The hybrid particles are suitable for implantable, depositable and or injectable delivery systems for sustained delivery of therapeutic active ingredients. Moreover, the particles can also be used for inhalation or oral delivery of therapeutic active substances.

The lipid/polymer hybrid particles contain at least one biodegradable polymer. The polymer can be a homopolymer, a random block copolymer or a mixture thereof. The polymer is partly or completely soluble in organic solvents and is not completely soluble in water. A biodegradable material is degraded to low molecular weight and may or may not be eliminated from the living organism. A biodegradable polymer can, for instance, be built from biodegradable monomer units. Example of homopolymers that is created from such monomers are: poly(lactide), poly(glycolide), poly(p-dioxanone), poly(caprolactone), polyhydroxyalkanoate, polypropylenefumarate, polyorthoesters, polyphosfateesters and polyanhydrides. Combinations (e.g. random or block) of these homopolymers can be used as well. Examples of copolymers are: different poly(D,L-lactide-co-glycolide) polymers or other biodegradable, or biocompatible, copolymers.

The lipid/polymer hybrid particles contain at least one lipid, or another amphifiilic molecule, with the ability to form the desired bilayer-containing or monolayer-containing phase in aqueous environments. The desired phase could for instance be cubic, sponge, lamellar, hexagonal, micellar or vesicular. Both synthetic and natural polar lipids (or other amphifilic molecules) can be used. The amphiphilic molecule can be anionic, cationic, zwitterionic or uncharged.

One suitable lipid for use in the present invention is an uncharged monoglyceride, called monoolein or glycerylmonooleate, which is polar but has the ability to swell, and which spontaneously forms a cubic bicontinuous phase in excess water. The cubic phase has a thermodynamically stable structure, where the lipid bilayer separates two almost identical water-channel systems. When consider the cubic phase in excess water, the only difference between the two water-channel system, is that one of them is connected with the outer water phase but the other one is not.

Examples of other lipids of interest, with the ability to form a cubic phase, are: monoelaidin, phosphatidylethanolamine, phospholipids and PEGylated phospolipids.

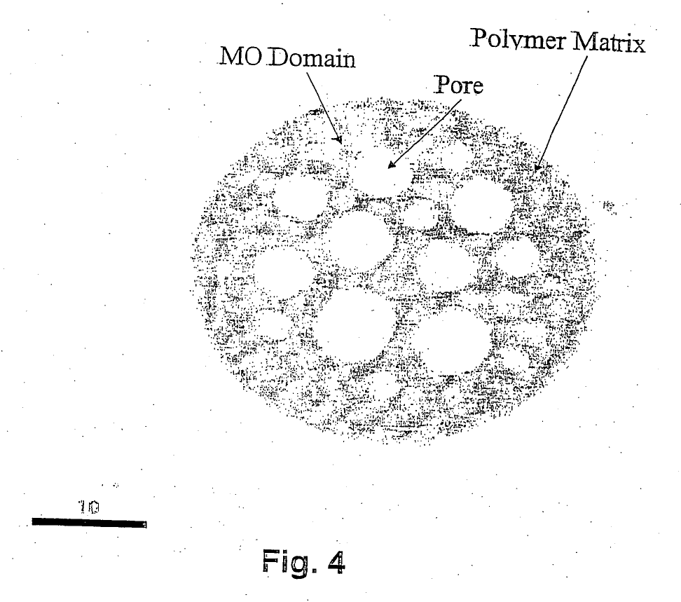

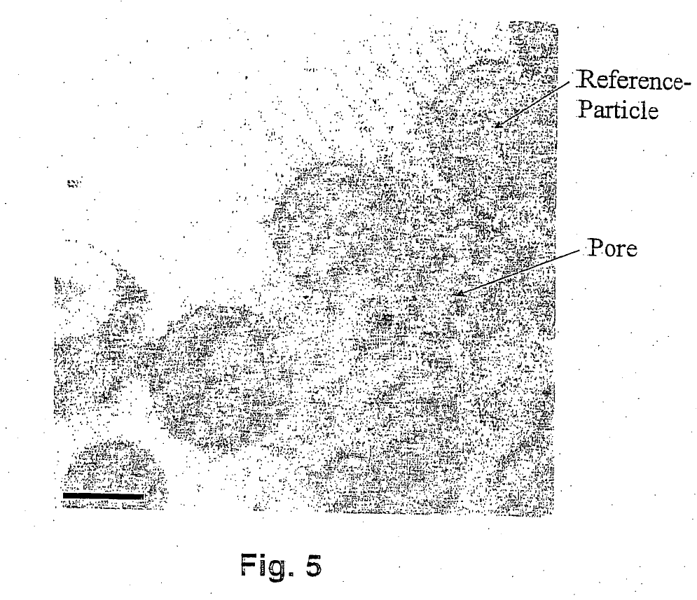

FIGS. 4 and 5 presents pictures from the characterization of the particles with CLSM, according to Example 2. The results showed that lipid domains were present in the lipid/polymer composite particles but not in the reference particles. Therefore, the domains found in the hybrid particles consist of MO.

When MO is exposed to water, the MO domains, located inside the particles, begin to swell. From the phase behavior of the EtAc/MO/water system it is noticeable that the lipid, as a result of water swelling (i.e. incorporation of water), first forms a swollen liquid phase. As a result of further swelling, the swollen liquid phase is transformed into the cubic phase(s).

By varying the composition of the outer phase the kinetics of the domain formation can be controlled. In the case with 100% water as outer phase, MO separated immediately from PLG-PEG (in less than 10 minutes from the formation of the particles). With 4% EtAc in the outer phase, MO separated and formed domains after approximately 10 minutes. When 6% EtAc was used in the outer phase, MO formed domains after around 16 minutes (smaller particles). In the case with an outer phase saturated with EtAc (8% EtAc in water), no domains had formed even after 90 minutes.

In all experiments, where the phase separation kinetics is slow, i.e. when the outer phase contain 6% or 8% EtAc, it is possible to detect large pores inside the emulsion droplets. The pores, presented in FIG. 4, are typically 3-9 μm. Later on, as a result of solvent removal, lipid domains are formed. Interestingly, the lipid domains start to swell from some of these pores. Hence, it seems reasonable that some pores contain a water-rich phase.

When the kinetics of the phase separation is rapid, i.e. when the outer phase consists of pure water, no such pores are observed. Instead, the swollen lipid domains are formed immediately. However, common for all cases with domain formation, irrespective of slow or rapid phase separation, is that the domains start to swell before they are rejected from the particles.

The particles are poly-disperse (1-100 μm) and MO has a tendency to form domains quicker in smaller particles than in larger. In fact, the smaller particles display a larger surface to volume ratio and thus they exhibit higher solvent removal efficiency. The particles examined by means of CLSM are, due to practical reasons (i.e. immobilization of the particles), 15-35 μm in diameter. However, particles of 1-10 μm size can easily be formed, which is a far more monodisperse distribution than in the present case.

Incorporation of Therapeutic Agents in the Lipid/Water Domain(s)

In the case of hydrophilic therapeutically active substances, it is possible to dissolve the active component into a small volume of water (e.g. 100 μl) before adding the L-phase, defined in Example 1. If the water content is small (i.e. low water activity) the L-phase will still be one true solution. However, the incorporation of water in the L-phase will also affect (i.e. induce) the phase separation of MO and PLG. If the water activity is high, MO and PLG will form two separate phases. If so, it will be possible to emulsify the lipid phase into the polymer rich phase. By emulsifying the lipid/polymer emulsion into an outer water-rich phase particles are formed as a consequence of solvent removal.

In the case of hydrophobic therapeutically active substances, the substances can be added directly to MO. The other components, i.e. polymer and solvent, are then added until the L-phase is formed.

Alternative Formulations

Other structures than particles can be made, e.g. solid implants and semi-solid, gel-like matrices. By using a water-insoluble polymer the former can be formed and by using a slightly water-soluble polymer the latter will form.

The form of the final product, i.e. particles, surfaces, implants etc., depends on the constitution of the polymer-rich phase while solvent removal, e.g. in aqueous solution, takes place. One obvious example is the formation of particles, where the polymer first is dissolved within the spherical emulsion droplets. Later on, as a consequence of solvent removal, particles are formed due to polymer precipitation.

The chemical system here presented is useful for formation of different kinds of particles (some are further presented below); particles with swelling lipid domains dispersed within the polymer matrix, particles with a lipid (or polymer) core placed within a polymer (or lipid) shell and porous polymer particles for inhalation applications. In addition, the chemical system might also be useful for formation of solid and semi-solid matrices, e.g. for depositable applications.

Applications of the Invention

In general, the invention is suitable for applications such as encapsulation of therapeutically active substances (e.g. proteins, peptides and antigens).

Due to the muco-adhesive property of MO the lipid/polymer composite particles are useful for oral delivery. However, for protein delivery other formulations can be useful as well.

In another aspect of the present invention the chemical system is suitable for functional food applications. Within the present application, functional food refers to physiologically active food, for instance food with a medical and/or prophylactic effect or food with a performance improvement effect.

In a further aspect of the present invention the chemical system is suitable for tissue engineering applications. One example is for formation of biodegradable polymer/lipid Schaffolds, which can be used for in vivo implantation. Living cells are then encapsulated into polymer or lipid environments. Insulin producing cells are one interesting example. Another example is so-called smart biodegradable polymer/lipid composite materials. Where the “smart” composite material can be used for delivery of active components to site-specific targets.

In still a further aspect of the present invention, the different environments, present within the composite material, can be used as media for chemical reactions. One interesting example is enzymatic reactions where the enzyme can be located in one compartment and the substrate in another compartment. The reaction will then take place in the vicinity of the interface between the different compartments.

In yet another aspect of the present invention, the different environments, present within the composite material, can be used for encapsulation of multiple components, e.g. for combo therapies, which can be released under different release rates.

One more specific application is to encapsulate calcitonin into the formulation, which, for instance, can be made to be injectable or depositable implants. The problem with calcitonin, as well as with other peptides, is to retain the activity during encapsulation, a problem that probably is eliminated or ameliorated with the invention. Examples of other interesting candidates for encapsulation are; growth hormone, insulin, oxytocin, parathyroid hormone, vasopressin.

Another specific application is DNA delivery. In this particular case it seems most fruitful to use a cationic amphiphilic molecule with the ability to form a lamellar phase in excess water. The reason, which already is known, is that the lamellar phase is suitable for DNA packing. The concept is useful for transfections in general. In addition, the concept can be used for delivery of DNA vaccines.

The porous polymer particles, described below, might be useful for applications such as inhalation.

The composite material includes, but is not limited to particles and implants. The depositable implants can be produced according to the following two-step process:

First the L-phase, containing polymer (PLG)/amphiphilic molecule (MO)/solvent(s) (EtAc) is formed. The active component is added in accordance with the procedure described above “Incorporation of Therapeutic Agents in the Lipid/water Domain(s)”. The one-phase region of the ternary PLG/MO/EtAc system, presented in the phase diagram, given in FIG. 1, define possible compositions of the L-phase. It is possible to vary the concentration of MO and PLG in a broad composition range (i.e. the MO can be varied from 0-15% by weight and the PLG content can approximately be varied between 0-100%). However, the resulting viscosity of the L-phase can be high if the L-phase is extremely rich in PLG (i.e. depending on polymer specific properties such as glass transition temperature and molecular weight).

The solvent is removed in the second step. The L-phase is then filled into a desired form and placed at a suitable temperature (which temperature depends on properties of both the solvent and therapeutically active component) until solvent removal has occurred. The product, consisting of a polymer-rich matrix containing domain(s) of the amphiphilic compound, can be useful for implant applications.

In addition, it seems also possible to produce in situ forming implants from the same system if using a non-toxic solvent. It might also be possible to inject a (warm) mixture containing therapeutically active component/amphiphilic molecule/polymer. It seems reasonable that the mixture, in some cases, also should contain water (e.g. when the therapeutically active component is hydrophilic).

Drug Release Mechanism

All formulations, here presented, could be useful for delivery of therapeutic active components with desired sustained/controlled release properties. The drug release mechanism will depend on a number of properties, some of here listed:
(i) The release of active component will be affected by the hydrophilic/hydrophobic properties of the active component. In fact, the active component will be positioned in different domains, within the cubic phase, depending on molecular structure. For instance, hydrophilic components are preferentially located in the water channels and hydrophobic substances are incorporated in the bilayer. In addition, components that exhibit surface-active properties are preferentially located at the interface between the two domains.
(ii) The diffusion of the active component depends on the molecular weight.
(iii) The hydrolysis, and water uptake, depends on some specific properties of the polymer, i.e. architecture, lactide/glycolide ratio, molecular weight, degree of pegylation in the case of pegylated polymer, as well as on temperature and pH.

In summary, different release profiles can be obtained depending on, for instance, type of formulation, active component, polymer properties and type of liquid crystalline phase, or monolayer-containing phase, formed.

Formation and Characterization of Lipid/Polymer Composite Particles

Now the method of the manufacture of particles, according to invention, will be described. The principle behind the method, according to the present invention, is to remove the solvent in a controlled manner, from a mixture or solution of a well defined starting solution, based on a quaternary phase diagram as discussed above. The starting formulation is either a one-phase solution or a two-phase sample, of a polymer and a lipid in a suitable solvent. Preferably, the starting sample is a true solution in one phase.

The removal of the solvent is performed, in one embodiment of the invention, by a process that could be regarded as a solvent extraction against a second solvent, in which the polymer is only slightly soluble or insoluble, whereby the polymer will precipitate in contact with the second solvent. The second solvent can preferably be water, or lower (cyclo)alkanes, e.g. cyclohexane, and esters.

Alternatively, a spray drying principle can be used, wherein the solution or mixture is sprayed such that the solvent evaporates, whereby the polymer will immediately precipitate in dry form. Supercritical fluid techniques can be used as well.

The polymer should have certain properties, namely it should not be completely miscible in the second solvent, and preferably it should be insoluble therein. At least, it must be possible to precipitate the polymer in the presence of the second solvent.

### EXAMPLES

### Example 1

### Manufacture of Lipid/Polymer Composite Particles

Lipid/polymer composite particles, presented in FIG. 4, with lipid domains dispersed in a polymer matrix, were formed according to the following procedure where a liquid phase (L-phase) was emulsified into a water-rich phase.

The following procedure was used for formation of the L-phase: The lipid probe, Lissamine™ rhodamine B 1,2-dihexadecanoyl-sn-glycero-3-phosphoethanolamine triethylammonium salt (rhodamine DHPE), was first dissolved in 95% ethanol (0.036 mg/ml). 0.59 g of the Rhodamine-DHPE solution was mixed with 0.063 g MO. The mixture was left at 50° C. until the ethanol had evaporated. Polymer (0.177 g) and solvent (2.70 g) were then added until the final composition of the L-phase became 2.1/6.0/91.9 MO/PLG/EtAc % by weight.

The L-phase, used for formation of reference particles, presented in FIG. 5, contained the same amounts of DHPE-probe, PLG and EtAc as the L-phase used for formation of lipid/polymer/composite particles.

Particles were formed by emulsifying 0.1 ml L-phase into 1.16 g outer phase, that consisted of 0%, 4%, 6%, or 8% EtAc in water. Both phases were room-temperatured when the emulsion was created by means of a Polytron mixer (Kinematica AG, PT300) working for 3 minutes at 4000 rpm. The particles were then spontaneously formed as a result of solvent removal.

### Example 2

### Particle Characterization

The particles were characterized by means of confocal laser scanning microscopy, CLSM. The emulsion/dispersion formed was inserted into an “in-house manufactured” chamber that in the bottom had a 17 μm thick cover glass. Onto the cover glass a glass cylinder (diameter) was glued with UV cured glue. The particles were immobilized by a monolayer of 0.2 mm glass beads, purchased from Kebo. The particles were examined by means of a CLSM (Leica TCS, 4D, Leica LT, Heidelberg, Germany) at room temperature with a Leica 63x/1.2 water immersion objective. The 568 nm line of the microscopes argon/krypton laser was used for excitation of rhodamine. The scanning was performed with 8 lines average and the resolution was set to XYZ (0.12 μm/0.12 μm/0.36 μm). The pictures, some of them containing both laser reflection (green) and rhodamine-DHPE probe emission (red), were processed using Image Space software (Molecular Dynamics, Sunnyvale, Calif.) on a workstation (Silicon Graphics, Mountain View, Calif.).

### Example 3

### Blood Compatibility Studies of Lipid/Polymer Composite Particles

The lipid/polymer hybrid particles have been characterized with respect to coagulation and hemolyse. Briefly, the experiments were carried out as follows:

Coagulation: Solvent/water mixture, with the same ratio used for particle formation, was used as negative control. Other samples were pure polymer, pure lipid, lipid/polymer composite particles and Taxol. Taxol was used as positive control. All experiments were carried out in dublicate.4.0 ml blood were added to each sample before the samples were circulating, at 37 degrees C. for 60 minutes, in tubes with the inside coated with heparin. The results showed no coagulation in any of the lipid-, polymer-, or composite sample.

Hemoluse: The samples were mixed with EDTA (form complexes with Ca²⁺). The blood plasma was separated from the red blood cells by 15 minutes centrifugation at 4 degrees Celcius. The lipid/polymer composite particles did not induce hemolyse.

Particles with Core/Shell Structure

By varying the lipid/polymer ratio, it is possible to create a core/shell structure where the lipid (or the polymer) forms the core within the polymer (or lipid) shell. In such formulation, the amount of lipid can be varied compared to the formulation described in Example 1.

Porous Polymer Particles

It is possible to produce very porous polymer particles according to the invention. These particles is interesting for inhalation since they, due to their porous interior structure, exhibit the same aerodynamic properties as smaller particles. The porous particles are created according to a two-step process. In the first step, lipid/polymer hybrid particles are created according to the detailed description given in Example 1. In the second step, the swollen lipid domains are washed out from the polymer matrix, by using a solvent that acts as a solvent for the lipid but not for the polymer. The final degree of porosity depends on the amount of lipid present. It should be possible to create particles with a degree of porosity of approximately 5-70%. The final polymer particles, when formed exhibit a very porous interior structure. Such particles are, after addition of a proper therapeutic active component, useful for inhalation applications.

1. A method of making a composite material, as well as the material itself, wherein the material contains at least one amphiphilic component and at least one polymer component, the method comprising the following steps:
providing a chemical system comprising the components of at least one polymer, at least one amphiphilic component and a (volatile) solvent or solvent mixture, wherein
i) the polymer is a homopolymer, a random-, or block-, copolymer or a mixture thereof; and
ii) the amphiphilic component has the ability to form a bilayer- or monolayer-containing phase; and
by use of a phase diagram, that graphically defines how the components of the chemical system interact in thermodynamically stable phases as a function of temperature, concentration and pressure, removing the solvent(s) from the chemical system by shifting the thermodynamic equilibrium point of said system in a controlled direction based on the phase diagram, thereby obtaining the desired material.

2. The method as claimed in claim 1, wherein the step of removing solvent comprises solvent extraction against a liquid phase containing at least one second solvent.

3. The method as claimed in claim 2, wherein the (volatile) solvent is not completely miscible with said second solvent.

4. The method as claimed in claim 2, wherein the second solvent is water or lower (cyclo)alkanes, such as e.g. cyclohexane or esters.

5. The method as claimed in claim 2, wherein the amphiphilic component/polymer mixture is an emulsion, and the emulsion is injected into an outer second solvent rich-phase, whereby particles are formed as a consequence of solvent removal.

6. The method as claimed in claim 1, wherein the step of removing solvent comprises spraying the mixture, so as to evaporate the solvent.

7. The method as claimed in claim 1, wherein the composite material obtained is one of particles, solid implants, semi-solid, gel-like matrices, or applied for surface coatings.

8. The method as claimed in claim 1, wherein the bilayer- or monolayer-containing phase is in the solid (crystalline) state or arranged in liquid (crystalline) phases such as cubic, sponge, lamellar, hexagonal, micellar or vesicular.

9. The method as claimed in claim 1, wherein the amphiphilic component is selected from synthetic and/or natural polar lipids or other amphiphilic components.

10. The method as claimed in claim 1, wherein the amphiphilic component is anionic, cationic, zwitterionic or uncharged.

11. The method as claimed in claim 1, wherein the amphiphilic component is selected from components having the ability to form a cubic, sponge, lamellar, hexagonal, micellar, or vesicular phase.

12. The method as claimed in claim 1, wherein the amphiphilic component is an uncharged monoglyceride, preferably glycerylmonooleate.

13. The method as claimed in claim 1, wherein the amphiphilic component is selected from monoelaidin, phosphatidyl-ethanolamine, phospholipids and PEGylated phospholipids or sfingolipids, cholesterol, brain- or skin lipids, or other lipid (or amphiphilic component) with the ability to form desired phase.

14. The method as claimed in claim 1, wherein the polymer is partially or completely soluble in organic solvents but not completely soluble in the second solvent.

15. The method as claimed in claim 1, wherein the polymer is a homopolymer selected from poly(lactide), poly(glycolide), poly(p-dioxanone), poly(caprolactone), polyhydroxyalkanoate, polypropylenefumarate, polyorthoesters, polyfhosphateesters and polyanhydrides, and combinations of these homopolymers, also modified by e.g. PEG.

16. The method as claimed in claim 1, wherein the polymer is a random or block-copolymer selected from different poly(D,L-lactide-co-glycolide) polymers or other biodegradable or biocompatible copolymers.

17. The method as claimed in claim 1, wherein the volatile solvent is partially miscible or insoluble with water.

18. Use of the material obtained by the method as claimed in claim 1, in implantable, depositable and or injectable delivery systems for sustained delivery of therapeutic active ingredients.

19. Use of material obtained by the method as claimed in claim 1, for functional food applications or applications e.g. site specific targets.

20. Use of material obtained by the method as claimed in claim 1, for making a formulation, e.g. particles, for inhalation or oral delivery of therapeutic active substance(s).

21. Composite material, comprising a polymer matrix exhibiting at least one domain comprising liquid (crystalline) phase or monolayer phase, said domain is dispersed within or on the surface of the polymer matrix (core/shell).

22. Material as claimed in claim 21, wherein said domains have a micellar or vesicular structure containing at least one second solvent, said structures being located inside said polymer matrix, optionally within voids.

23. Material as claimed in claim 21, in the form of (nano/micro) particles.

24. Material as claimed in 23 claim 21, in the form of solid implants, semi-solid, gel-like matrices, or applied for surface coatings.

25. A concept for either sustained/controlled release of therapeutically active component, or for prolonged/retained activity of sensitive therapeutically active component.
